(19) United States
(12) Patent Application Publication
(10) Pub. No.: US 2019/0000761 A1
(43) Pub. Date: Jan. 3, 2019

(54) METHODS OF TREATING ACNE USING NANOEMULSION COMPOSITIONS

(71) Applicant: NanoBio Corporation, Ann Arbor, MI (US)

(72) Inventors: Joyce SUTCLIFFE, Ann Arbor, MI (US); Susan CIOTTI, Ann Arbor, MI (US); James R. BAKER, Ann Arbor, MI (US)

(73) Assignee: NanoBio Corporation, Ann Arbor, MI (US)

(21) Appl. No.: 15/748,625

(22) PCT Filed: Jul. 29, 2016
(86) PCT No.: PCT/US2016/044712

§ 371 Date: Jan. 29, 2018

Related U.S. Application Data
(60) Provisional application No. 62/199,426, filed on Jul. 31, 2015.

(51) Int. Cl. A61K 9/107; A61K 31/203; A61K 31/327; A61K 31/60; A61K 47/18; A61K 47/26; A61K 47/44; A61K 9/00; A61P 17/10

(52) U.S. Cl. CPC A61K 9/1075; A61K 31/203; A61K 31/327; A61K 31/60; A61P 17/10; A61K 47/183; A61K 47/26; A61K 47/44; A61K 9/0014; A61K 47/186

(57) ABSTRACT

The present invention relates to methods for treating and preventing acne or P. acnes infection in a subject comprising topically administering to the subject in need thereof an anti-acne nanoemulsion composition, comprising CPC or BKC as a cationic agent.

### CROSS-REFERENCE TO RELATED APPLICATION

This application claims priority of U.S. Provisional Application No. 62/199,426, filed on Jul. 31, 2015, the contents of which are specifically incorporated by reference.

### FIELD OF THE INVENTION

The present disclosure relates to improved compositions and methods for preventing, and/or treating acne or killing, and/or inhibiting the growth of Propionibacterium acnes. The method comprises topically administering to a subject in need thereof a nanoemulsion composition comprising cetyl pyridium chloride, benzalkonium chloride, or a combination thereof, at preferred concentrations, where the nanoemulsion has anti-acne properties.

### BACKGROUND OF THE INVENTION

### A. Acne and P. acnes Infection

Acne is a chronic inflammatory disease affecting more than 85% of teenagers, and continuing into adulthood in some populations. Some individuals suffer from acne into their thirties, forties and beyond. Acne is most frequently found on the face and upper neck, but also found on the chest, back, shoulders and upper arms. Acne lesions can develop into comedo, papule, pustule, lupus, nodule, or scars.

Acne is a disease of pilosebaceous units in the skin. Although the cause of acne is not fully understood, some factors have been linked to acne, such as genetic history, hormone level, skin inflammation, etc. In acne, excessive sebum production occurs in the sebaceous gland. This causes hyperkeratinization of the hair follicle and prevents normal shedding of the follicular keratinocytes. This results in obstruction of the hair follicle and subsequent accumulation of lipids and cellular debris in the blocked hair follicle. Colonization of an anaerobic gram-positive bacterium, Propionibacterium species, e.g., Propionibacterium acnes, occurs in the blocked follicle. This bacteria is present on most human skin and lives on fatty acids in the pilosebaceous unit. Infection of the hair follicle results in inflammation. Inflammation is further enhanced by rupture of the hair follicle and release of lipids, bacteria, and fatty acids into the dermis.

P. acnes is one of the primary factors involved in the pathogenesis of acne vulgaris. It is the predominant microorganism of the pilosebaceous glands of human skin, with up to 10 million viable organisms isolated from a single sebaceous unit. Although aerotolerant, P. acnes typically grows in the anaerobic environment of the infrainfundibulum, where it releases lipases and digests local accumulations of the skin, oil and sebum.

### B. Conventional Treatment for Acne

Conventional treatment for acne includes topical or oral administration of bactericidals, benzoyl peroxide, triclosan bekeratolytics, e.g., salicylic acid, and chlorhexidine, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, retinoids, e.g., tretinoin, isotretinoin, adapalene and tazarotene, antibiotics, e.g., erythromycin, clindamycin, tetracycline, minocycline, doxycycline, hormones, e.g., estrogen, and progesterone, and combination products, e.g., stievamycin, Murad®, Benzaclin® and Benzamycin®. Other anti-acne ingredients include Ascorbyl Tetraisopalmitate, Dipotassium Glycyrrhizinate, Ascorbyl Tetraisopalmitate, Niacinamide, alpha bisabolol. All of these ingredients have properties that help to reduce and control acne, and acne related problems such as sebum production. Herbal medicines are also used to treat acne and include Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil. These various treatments for acne may have only temporary effects, and may cause drug-resistance or other undesirable side effects, such as allergy, skin redness, or skin hypersensitivity.

Orally administered drugs are generally more effective than topically applied drugs, but because they act systemically rather than locally, the side effects of orally administered drugs can limit their use.

### C. Background Regarding Nanoemulsions

Prior teachings related to nanoemulsions are described in U.S. Pat. No. 6,015,832, which is directed to methods of inactivating a Gram-positive bacteria, a bacterial spore, or a Gram-negative bacteria. The methods comprise contacting the Gram-positive bacteria, bacterial spore, or Gram-negative bacteria with a bacteria-inactivating (or bacterial-spore inactivating) emulsion. U.S. Pat. No. 6,506,803 is directed to methods of killing or neutralizing microbial agents (e.g., bacteria, virus, spores, fungus, on or in humans using an emulsion. U.S. Pat. No. 6,559,189 is directed to methods for decontaminating a sample (human, animal, food, medical device, etc.) comprising contacting the sample with a nanoemulsion. U.S. Pat. No. 6,635,676 is directed to two different compositions and methods of decontaminating samples by treating a sample with the compositions. U.S. Pat. No. 7,314,624 is directed to methods of inducing an immune response to an immunogen comprising treating a subject via a mucosal surface with a combination of an immunogen and a nanoemulsion. US-2005-0208083-A1 and US-2006-0251684-A1 are directed to nanoemulsions having droplets with preferred sizes. US-2007-0054834-A1 is directed to compositions comprising quaternary ammonium halides and methods of using the same to treat infectious conditions. US-2007-0036831-A1 is directed to nanoemulsions comprising an anti-inflammatory agent.

Finally, US-2010-0226983-A1 broadly describes nanoemulsion compositions for treatment and prevention of acne. The present invention is an improvement over this earlier disclosure. In particular, prior to the present invention it was not known that (1) the presence of a cationic agent in a nanoemulsion is critical to successful treatment of acne, (2) the identity of the cationic agent is important; and (3) an effective nanoemulsion for treatment of acne requires a narrow concentration range specific to a cationic agent.

There is a need in the art for improved treatment and prevention options for patients affected by acne. Specifically, there is a need in the art for an effective topical agent to treat and prevent acne and/or infection by P. acnes. The present invention satisfies these needs.

### SUMMARY OF THE INVENTION

The present invention provides methods and compositions for treating and/or preventing acne and/or infection by P. acnes in a subject comprising administering a nanoemulsion topically to the subject, wherein the nanoemulsion comprises as a cationic surfactant cetylpyridinium chloride (CPC), benzalkonium chloride (BKC), or a combination thereof. If CPC is present as the cationic surfactant, then CPC is present at a concentration range in the undiluted nanoemulsion of from about 0.10% to about 0.40%. If BKC is present as the cationic surfactant, then BKC is present at a concentration range in the undiluted nanoemulsion of about 0.05% to about 1.6%. As a reference point, a 0.20% concentration of CPC correlates with a 0.80% concentration of BKC (although a slightly lower concentration than this correction has been found to be effective; e.g., this correction suggests a bottom threshold of 0.4% BKC, but a lower amount of 0.13% has been found to be effective in studies).

The nanoemulsion comprises droplets having an average diameter of less than about 3 microns, and the nanoemulsion droplets comprise an aqueous phase, at least one oil, at least one cationic surfactant which is CPC or BKC, and at least one organic solvent.

Surprisingly, it was discovered that the topically applied nanoemulsions comprising the specified concentration of CPC or BKC exhibit dramatically increased effectiveness in vivo in reducing the presence of P. acnes, as described in the examples below.

The nanoemulsions of the invention exhibit cidal activity against P. acnes and synergy with other agents commonly used to treat acne. The composition of the invention allow for targeted delivery into the pilosebaceous unit, the site of acne pathogenesis. This is significant, as a topically applied, and therefore local, site-specific activity, is highly preferable over an orally administered, and therefore systemic activity. Moreover, the nanoemulsions are able to enhance delivery, and thus effectiveness, of other topical anti-acne agents incorporated into the nanoemulsion, thereby enhancing the efficacy and reducing the detrimental side effects of the other anti-acne agents.

In other embodiments of the invention, the nanoemulsion at the time of topical application is at room temperature or warmer. Optionally, the nanoemulsion can further comprise a chelating agent. In one embodiment of the invention, the nanoemulsion used in the methods of the invention can be diluted. The diluted samples can then be tested to determine if they maintain the desired functionality, such as surfactant concentration, stability, particle size, and/or anti-infectious activity (e.g., antimicrobial activity against P. acnes).

In some embodiments, a second anti-acne agent is incorporated into the nanoemulsion to achieve improved delivery, efficacy and or tolerability of the second anti-acne agent. Any suitable or desirable second active agent useful in treating acne can be incorporated into the nanoemulsion of the invention. Inclusion of a second anti-acne agent into the nanoemulsion should reduce the potential for resistance development towards either the nanoemulsion or added anti-acne agent. The nanoemulsion may further comprise anti-comedogenic, anti-inflammatory, keratolytics, sebum suppressors as disclosed in PCT publication No. WO/01/56556 A2.

The nanoemulsions for topical administration can be in the form of any pharmaceutically acceptable dosage form, including but not limited to, ointments, creams, emulsions, lotions, gels, liquids, bioadhesive gels, sprays, shampoos, aerosols, pastes, foams, sunscreens, capsules, microcapsules, or in the form of an article or carrier, such as a bandage, insert, syringe-like applicator, pessary, powder, talc or other solid, shampoo, cleanser (leave on and wash off product), and agents that favor penetration within pilosebaceous unit, the epidermis, the dermis and keratin layers. The nanoemulsion is capable of effectively treating, preventing, and/or curing acne, without being systemically absorbed and without significantly irritating the skin.

The foregoing general description and following brief description of the drawings and the detailed description are exemplary and explanatory and are intended to provide further explanation of the invention as claimed. Other objects, advantages, and novel features will be readily apparent to those skilled in the art from the following detailed description of the invention.

### BRIEF DESCRIPTION OF THE DRAWINGS

FIG. 1 illustrates the cross-section view of the pilosebaceous unit in human cadaver skin and hamster ear after application of nanoemulsion plus fluorescein

FIG. 2 shows the bactericidal effect on pig skin inoculated with P. acnes. The figure shows log reduction of P. acnes bacterial colonies after 1 hr of treatment with four nanoemulsions differing in CPC concentration: 0.01% CPC, 0.1% CPC, 0.25% CPC, and 0.5% CPC. All concentrations of nanoemulsions tested showed similar bactericidal activity in the preclinical skin model.

FIG. 3 shows the net change in log/cm² of the total P. acnes/cm² on the forehead of human subjects during a 4 week study comparing the effect of the nanoemulsion NB-003 0.5%, comprising 0.5% CPC, and vehicle, in preventing acne.

FIG. 4 shows the net change in log/cm² of the total P. acnes/cm² on the forehead of human subjects during a 4 week study comparing the effect of the nanoemulsion NB-003 0.3%, comprising 0.3% CPC, and the nanoemulsion NB-003 0.1%, comprising 0.1% CPC.

FIG. 5 shows the log reduction of P. acnes over a 4 week period in human subjects with topical application to the forehead, twice daily, of a control or one of four different nanoemulsion formulations (0.1% NB-003; 0.25% NB-003; 0.30% NB-003; or 0.05% NB-003).

### DETAILED DESCRIPTION OF THE PREFERRED EMBODIMENTS

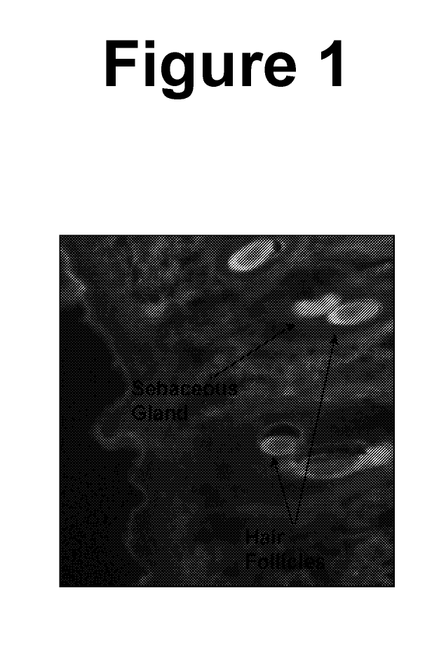

The present disclosure provides methods and compositions for treating, preventing, and/or curing acne and/or infection by P. acnes in a subject comprising administering topically to the subject a nanoemulsion comprising as a cationic surfactant cetylpyridinium chloride, benzalkonium chloride, or a combination thereof. The nanoemulsion further comprises droplets having an average diameter of less than about 3 microns, and the nanoemulsion droplets comprise an aqueous phase, at least one oil, at least one surfactant, and at least one organic solvent. The delivery of nanoemulsions is targeted to the site of acne pathogenesis. i.e., the pilosebaceous unit. See FIG. 1.

Since nanoemulsions are not a single small molecule, their relative activity can be expressed in terms of the concentration of cationic surfactant present, e.g., CPC or benzalkonium chloride. Thus, a “0.3%” nanoemulsion refers to the concentration of CPC or BKC in the nanoemulsion.

As noted above, US-2010-0226983-A1 broadly describes nanoemulsion compositions for treatment and prevention of acne. The present invention is an improvement over this earlier disclosure. In particular, prior to the present invention it was not known that the presence of a cationic agent in a nanoemulsion is critical to successful treatment of acne, nor was it known that the concentration of the cationic acid is specific to a particular agent. Additionally, it was not known that conventional pig skin models for evaluating percutaneous drug absorption are not predictive of in vivo efficacy for topically applied nanoemulsions for treating and/or preventing human acne.

Specifically, US-2010-0226983-A1 teaches that a wide concentration range of various surfactants can be utilized in nanoemulsions to be used to treat human acne, and in particular that high concentrations of CPC were particularly effective in treating or preventing human acne. In particular, US-2010-0226983-A1 teaches that nanoemulsions useful in treating and/or preventing acne comprise about 0.001% to about 10% surfactant or detergent, or a combination of at least one non-ionic surfactant present in an amount of about 0.1% to about 10% and at least one cationic agent present in an amount of about 0.01% to about 2% (paragraph [0033]). Additionally, US-2010-0226983-A1 teaches that high concentrations of CPC, such as 0.8% or higher “showed a dramatic increase in [skin] permeation” of the nanoemulsion (paragraph [0040], referring to data shown in FIG. 6 and Example 7 of US-2010-0226983-A1).

These teachings of US-2010-0226983-A1 are based on experimental data in the Examples, which notably, consists of in vitro data (Examples 2, 3, 5 and 8), including experimental skin permeation models using pig skin (Examples 4 and 8). Pig skin is a well-known animal model, and is taught in the art to be predictive of permeation across human skin. See B. Godin and E. Touitou, “Transdermal skin delivery: Predictions for humans from in vivo, ex vivo and animal models”, Advanced Drug Delivery Reviews, 59 (11):1152-61 (10/2007) (“the most relevant animal model for human skin is the pig. Porcine skin is readily obtainable from abattoirs and its histological and biochemical properties have been repeatedly shown to be similar to human skin . . . Porcine ear skin is particularly well-suited for permeation studies and gives comparable results to human skin.”)

More specifically, Example 7 of US-2010-0226983-A1 evaluates the in vitro permeation of the active present in a nanoemulsion into the dermis and epidermis of pig skin. Paragraphs [0188]-[0189]. Four different CPC concentrations of nanoemulsion were tested: 0.25%, 0.30%, 0.50% and 0.80%. Id. The skin permeation results utilizing pig skin epidermis, shown in FIGS. 6 and 7, show that higher concentration nanoemulsions result in significantly improved permeation, and the reference concludes that higher CPC concentration nanoemulsions, e.g. 0.8% CPC, “were found to deliver three times the amount of the surfactant, cetylpyridinium chloride (CPC) to the dermis as compared to a lower viscosity nanoemulsion,” e.g., 0.25% NB-003. Paragraphs [0189]-[0191] and [0040] of US-2010-0226983-A1.

The present invention is directed to the surprising discovery that conventional preclinical skin models, such as pig skin models, are not predictive of topical nanoemulsions useful in treating and preventing human acne.

Specifically, it was surprisingly discovered that topical nanoemulsions comprising as a cationic surfactant cetylpyridinium chloride, benzalkonium chloride, or a combination thereof, when present in specific concentration ranges, are effective in treating and/or preventing acne and/or infection by P. acnes in a human subject. The in vivo concentrations of CPC and BKC effective in treating and/or preventing acne detailed in the present invention do not correlate with in vitro efficacy in killing P. acnes. Moreover, the in vivo concentrations of CPC and BKC effective in treating and/or preventing acne detailed in the present invention do not correlate with the concentrations of CPC and BKC in nanoemulsion compositions shown to be effective in killing P. acnes in pig skin models. This is particularly surprising as pig skin models were believed to be predictive of in vivo human skin permeations.

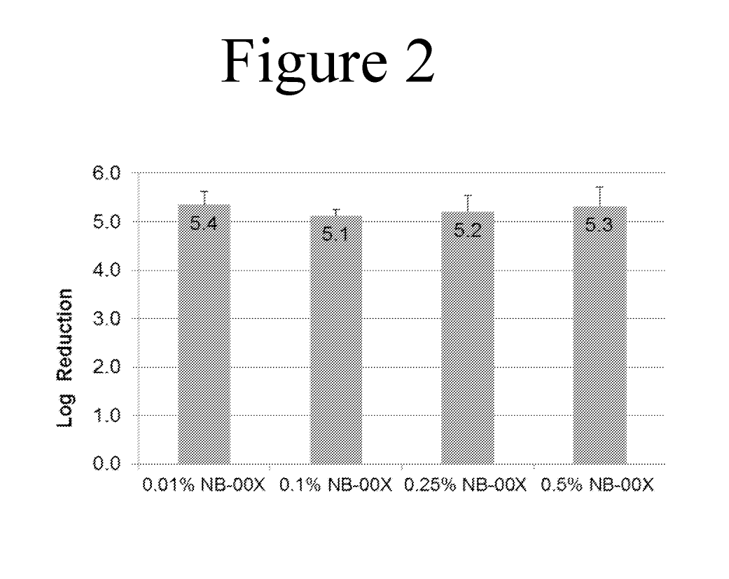

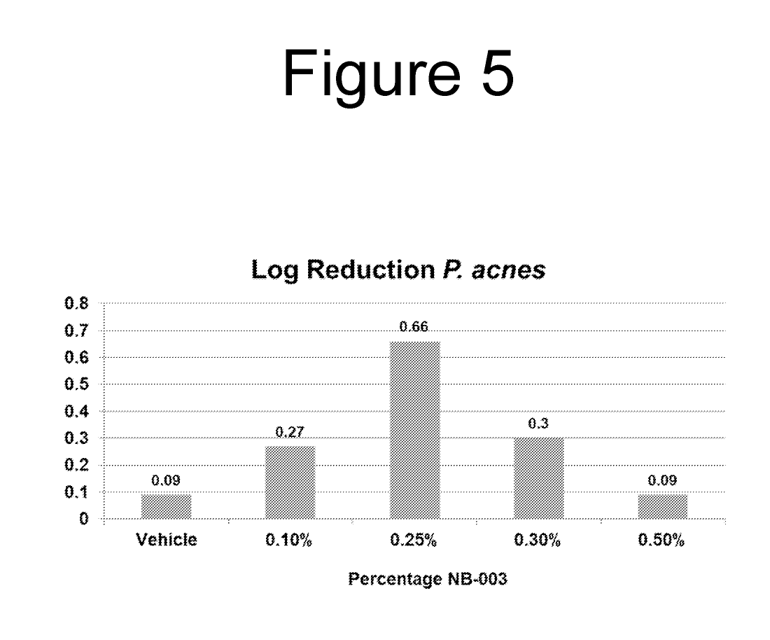

As described in the Examples below, in preclinical skin models using non-human skin, e.g., pig skin inoculated with P. acnes, all concentrations of nanoemulsions tested—0.01% CPC, 0.1% CPC, 0.25% CPC, and 0.5% CPC—were found to have high bactericidal activity. See FIG. 2 and Example 2 below. In stark contrast, however, these preclinical results were not predictive of the nanoemulsion formulations found to be effective in vivo in treating and/or preventing human acne. Specifically, FIG. 5 and Example 4 below show the results of a Phase 1 study. The study was a randomized, active-controlled, open label, single-site study in normal human volunteers with P. acnes colonization. The human in vivo results unexpectedly showed that bactericidal activity dramatically drops off at 0.5% CPC, with an optimal CPC concentration in a nanoemulsion for treatment and/or prevention of acne of between about 0.1% and about 0.4%.

### Concentrations of CPC and BKC

If cetylpyridinium chloride (CPC) is present as the cationic surfactant, CPC is present at a concentration range in the undiluted nanoemulsion of from about 0.1% to about 0.4%, or any amount in-between these two amounts. In other embodiments of the invention, the CPC concentration in the undiluted nanoemulsion is about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.26%, about 0.27%, about 0.28%, about 0.29%, about 0.30%, about 0.31%, about 0.32%, about 0.33%, about 0.34%, about 0.35%, about 0.36%, about 0.37%, about 0.38%, about 0.39%, or about 0.4%.

In nanoemulsions useful in the methods of treating and/or preventing acne according to the invention, a 0.20% concentration of CPC correlates with a 0.80% concentration of BKC; thus, a higher concentration of BKC is required for an effective nanoemulsion. However, a slightly lower concentration of BKC than this correction has been found to be effective; e.g., this correction suggests a bottom threshold of 0.4% BKC, but a lower amount of 0.13% has been found to be effective in studies. Thus, if benzalkonium chloride (BKC) is present as the cationic surfactant, then BKC is present at a concentration range in the undiluted nanoemulsion of about 0.05% to about 1.6%, or any amount in-between these two amounts. In other embodiments of the invention, BKC can be present at a concentration of about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.2%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.25%, about 1.3%, about 1.35%, about 1.4%, about 1.45%, about 1.5%, about 1.55%, or about 1.6%, or any amount inbetween these amounts.

In another embodiment of the invention, the nanoemulsion further comprises a chelating agent. The organic solvent and the aqueous phase of the invention can be a non-phosphate based solvent.

In one embodiment of the invention, the nanoemulsion comprises: (a) an aqueous phase; (b) about 0.1% to about 0.4% CPC, or about 0.05% to about 1.6% BKC; (c) about 1% to about 80% oil, or about 5% to about 80% oil; and (d) about 0.1% to about 50% organic solvent, or about 1% to about 10% organic solvent. Optionally, the nanoemulsion can further comprise about 0.0005% to about 1.0% of a chelating agent such as EDTA (but the chelating agent is not limited to EDTA) and/or a non-cationic surfactant, such as but not limited to a non-ionic surfactant, at a concentration of about 0.001% to about 10%, or about 0.1% to about 10%.

In one embodiment of the invention, the nanoemulsion further comprises at least one non-cationic surfactant. The non-cationic surfactant can be a nonionic surfactant, such as a polysorbate. Exemplary polysorbates are polysorbate 20, polysorbate 80, and polysorbate 60. In some embodiments, the non-ionic surfactant is present in a concentration of about 0.001% to about 10%; about 0.05% to about 7.0%; or about 0.5% to about 4%.

In some embodiments, an anti-acne agent is incorporated into the nanoemulsion to achieve improved delivery, efficacy and/or tolerability of the added anti-acne agent. Examples of suitable topical anti-acne agents include, but are not limited to, benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, tetracycline, doxycycline, minocycline, meclocycline erythromycin, clindamycin, azelaic acid, hydrocortisone, sodium hyaluronate, sulfur, urea, dapsone, adapalene, tretinoin, retinoids and retinoid-derived compounds. Other anti-acne ingredients include Ascorbyl Tetraisopalmitate, Dipotassium Glycyrrhizinate, Ascorbyl Tetraisopalmitate, Niacinamide, alpha bisabolol. All of these skin care ingredients have properties that help to reduce and control acne, and acne related problems such as sebum production. Examples of acne herbal medicines include, but are not limited to, Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil. The anti-acne active agent can be present in a therapeutically effective amount, such as from about 0.001% up to about 99%, about 0.01% up to about 95%, about 0.1% up to about 90%, about 3% up to about 80%, about 5% up to about 60%, about 10% up to about 50%, or any combination thereof (e.g., about 3% up to about 10%).

Viscosity: As described in U.S. Patent Publication No. 2010/0226983, the viscosity of the nanoemulsion can be increased to provide improved therapeutic effectiveness. Examples of methods of increasing the viscosity of a nanoemulsion according to the invention include adding a thickening agent or gelling agent to the formulation.

Thus, in one embodiment of the invention, the nanoemulsion has a viscosity of greater than about 12 centipoise (cP), greater than about 15 cP, greater than about 20 cP, greater than about 25 cP, greater than about 30 cP, greater than about 35 cP, greater than about 40 cP, greater than about 45 cP, greater than about 50 cP, greater than about 55 cP, greater than about 60 cP, greater than about 65 cP, greater than about 70 cP, greater than about 75 cP, greater than about 80 cP, greater than about 85 cP, greater than about 90 cP, greater than about 95 cP, greater than about 100 cP, greater than about 150 cP, greater than about 200 cP, greater than about 300 cP, greater than about 400 cP, greater than about 500 cP, greater than about 600 cP, greater than about 700 cP, greater than about 800 cP, greater than about 900 cP, greater than about 1000 cP, greater than about 1500 cP, greater than about 2000 cP, greater than about 2500 cP, greater than about 3000 cP, greater than about 3500 cP, greater than about 4000 cP, greater than about 4500 cP, greater than about 5000 cP, greater than about 5500 cP, greater than about 6000 cP, greater than about 7000 cP, greater than about 8000 cP, greater than about 9000 cP, greater than about 10,000 cP, greater than about 15,000 cP, greater than about 20,000 cP, greater than about 30,000 cP, greater than about 40,000 cP, greater than about 50,000 cP, greater than about 60,000 cP, greater than about 70,000 cP, greater than about 80,000 cP, greater than about 90,000 cP, greater than about 100,000 cP, greater than about 150,000 cP, greater than about 200,000 cP, greater than about 250,000 cP, or up to about 259,300 cP.

Temperature: In some embodiments, the effectiveness of a nanoemulsion according to the invention in treating acne can be improved by ensuring that the nanoemulsion is at room temperature or warmer prior to application.

Thus, in another embodiment of the invention, encompassed are methods of treating acne comprising application of a nanoemulsion according to the invention, wherein the nanoemulsion is at room temperature (e.g., 20 to 25° C.). In another embodiment of the invention, encompassed are methods of treating acne comprising application of a nanoemulsion according to the invention, wherein the nanoemulsion has been warmed prior to application. For example, the nanoemulsion can be warmed prior to application to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, about 37° C. or warmer,

### A. Definitions

The present invention is described herein using several definitions, as set forth below and throughout the application.

As used herein, “about” will be understood by persons of ordinary skill in the art and will vary to some extent depending upon the context in which it is used. If there are uses of the term which are not clear to persons of ordinary skill in the art given the context in which it is used, “about” will mean up to plus or minus 10% of the particular term.

The terms “buffer” or “buffering agents” refer to materials which when added to a solution, cause the solution to resist changes in pH.

The terms “chelator” or “chelating agent” refer to any materials having more than one atom with a lone pair of electrons that are available to bond to a metal ion.

The term “dilution” refers to dilution of the nanoemulsions of the present invention or those derived from the nanoemulsions of the present invention using, for example, an aqueous system comprised of PBS or water (such as diH₂O), or other water soluble components, to the desired final concentration.

The term “nanoemulsion,” as used herein, includes dispersions or droplets, as well as other lipid structures that can form as a result of hydrophobic forces that drive apolar residues (i.e., long hydrocarbon chains) away from water and drive polar head groups toward water, when a water immiscible oily phase is mixed with an aqueous phase. These other lipid structures include, but are not limited to, unilamellar, paucilamellar, and multilamellar lipid vesicles, micelles, and lamellar phases. The droplets have an average diameter of less than about 3 microns.

The terms “pharmaceutically acceptable” or “pharmacologically acceptable,” as used herein, refer to compositions that do not substantially produce adverse allergic or immunological reactions when administered to a host (e.g., an animal or a human). Such formulations include any pharmaceutically acceptable dosage form. As used herein, “pharmaceutically acceptable carrier” includes any and all solvents, dispersion media, coatings, wetting agents (e.g., sodium lauryl sulfate), isotonic and absorption delaying agents, disintegrants (e.g., potato starch or sodium starch glycolate), and the like.

The term “stable” when referring to a “stable nanoemulsion” means that the nanoemulsion retains its structure as an emulsion. A desired nanoemulsion structure, for example, may be characterized by a desired size range, macroscopic observations of emulsion science (is there one or more layers visible, is there visible precipitate), pH, and a stable concentration of one or more the components.

The term “subject” as used herein refers to organisms to be treated by the compositions of the present invention. Such organisms include animals (domesticated animal species, wild animals), and humans.

The term “surfactant” refers to any molecule having both a polar head group, which energetically prefers solvation by water, and a hydrophobic tail which is not well solvated by water. The term “cationic surfactant” refers to a surfactant with a cationic head group. The term “anionic surfactant” refers to a surfactant with an anionic head group.

As used herein, the term “topically” refers to application of the compositions of the present invention to the surface of the skin and tissues.

### B. Stability of the Nanoemulsions of the Invention

The nanoemulsions of the invention are stable at about 40° C. and about 75% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years.

In another embodiment of the invention, the nanoemulsions of the invention are stable at about 25° C. and about 60% relative humidity for a time period of at least up to about 1 month, at least up to about 3 months, at least up to about 6 months, at least up to about 12 months, at least up to about 18 months, at least up to about 2 years, at least up to about 2.5 years, or at least up to about 3 years, at least up to about 3.5 years, at least up to about 4 years, at least up to about 4.5 years, or at least up to about 5 years.

Further, the nanoemulsions of the invention are stable at about 4° C. for a time period of at least up to about 1 month, at least up to about 3 months, at least up to about 6 months, at least up to about 12 months, at least up to about 18 months, at least up to about 2 years, at least up to about 2.5 years, at least up to about 3 years, at least up to about 3.5 years, at least up to about 4 years, at least up to about 4.5 years, at least up to about 5 years, at least up to about 5.5 years, at least up to about 6 years, at least up to about 6.5 years, or at least up to about 7 years.

### C. Nanoemulsions

The term “nanoemulsion”, as defined herein, refers to a dispersion or droplet or any other lipid structure. Typical lipid structures contemplated in the invention include, but are not limited to, unilamellar, paucilamellar and multilamellar lipid vesicles, micelles and lamellar phases.

The nanoemulsion of the present invention comprises droplets having an average diameter size of less than about 3 microns, less than about 2500 nm, less than about 2000 nm, less than about 1500 nm, less than about 1000 nm, less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, or any combination thereof. In one embodiment, the droplets have an average diameter size greater than about 50 nm, greater than about 100 nm, or greater than about 125 nm, and at least 400 nm. In another embodiment, the droplets have an average diameter of about 180 nm.

### 1. Aqueous Phase

The aqueous phase can comprise any type of aqueous phase including, but not limited to, water (e.g., H₂O, distilled water, tap water) and solutions (e.g., phosphate-buffered saline (PBS) solution). In certain embodiments, the aqueous phase comprises water at a pH of about 4 to 10, preferably about 6 to 8. The water can be deionized (hereinafter “DiH₂O”). In some embodiments the aqueous phase comprises phosphate-buffered saline (PBS). The aqueous phase may further be sterile and pyrogen free.

### 2. Organic Solvents

Organic solvents in the nanoemulsions of the invention include, but are not limited to, C₁-C₁₂ alcohol, diol, triol, dialkyl phosphate, tri-alkyl phosphate, such as tri-n-butyl phosphate, semi-synthetic derivatives thereof, and combinations thereof. In one aspect of the invention, the organic solvent is an alcohol chosen from a nonpolar solvent, a polar solvent, a protic solvent, or an aprotic solvent.

Suitable organic solvents for the nanoemulsion include, but are not limited to, ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof.

### 3. Oil Phase

The oil in the nanoemulsion of the invention can be any cosmetically or pharmaceutically acceptable oil. The oil can be volatile or non-volatile, and may be chosen from animal oil, vegetable oil, natural oil, synthetic oil, hydrocarbon oils, silicone oils, semi-synthetic derivatives thereof, and combinations thereof.

Suitable oils include, but are not limited to, mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, Flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and any combinations thereof.

The oil may further comprise a silicone component, such as a volatile silicone component, which can be the sole oil in the silicone component or can be combined with other silicone and non-silicone, volatile and non-volatile oils. Suitable silicone components include, but are not limited to, methylphenylpolysiloxane, simethicone, dimethicone, phenyltrimethicone (or an organomodified version thereof), alkylated derivatives of polymeric silicones, cetyl dimethicone, lauryl trimethicone, hydroxylated derivatives of polymeric silicones, such as dimethiconol, volatile silicone oils, cyclic and linear silicones, cyclomethicone, derivatives of cyclomethicone, hexamethylcyclotrisiloxane, octamethylcyclotetrasiloxane, decamethylcyclopentasiloxane, volatile linear dimethylpolysiloxanes, isohexadecane, isoeicosane, isotetracosane, polyisobutene, isooctane, isododecane, semi-synthetic derivatives thereof, and combinations thereof.

The volatile oil can be the organic solvent, or the volatile oil can be present in addition to an organic solvent. Suitable volatile oils include, but are not limited to, a terpene, monoterpene, sesquiterpene, carminative, azulene, menthol, camphor, thujone, thymol, nerol, linalool, limonene, geraniol, perillyl alcohol, nerolidol, farnesol, ylangene, bisabolol, farnesene, ascaridole, chenopodium oil, citronellal, citral, citronellol, chamazulene, yarrow, guaiazulene, chamomile, semi-synthetic derivatives, or combinations thereof.

In one aspect of the invention, the volatile oil in the silicone component is different than the oil in the oil phase.

### 4. Surfactants/Detergent

The nanoemulsions of the invention comprise at least one cationic surfactant which is CPC, BKC, or a combination thereon, as described above. The nanoemulsion can additionally comprise one or more non-cationic surfactants, which can be a pharmaceutically acceptable ionic surfactant, a pharmaceutically acceptable nonionic surfactant, a pharmaceutically acceptable anionic surfactant, or a pharmaceutically acceptable zwitterionic surfactant.

Exemplary useful surfactants are described in Applied Surfactants: Principles and Applications. Tharwat F. Tadros, Copyright 8 2005 WILEY-VCH Verlag GmbH & Co. KGaA, Weinheim ISBN: 3-527-30629-3), which is specifically incorporated by reference.

Exemplary surfactants include, but are not limited to, ethoxylated nonylphenol comprising 9 to 10 units of ethyleneglycol, ethoxylated undecanol comprising 8 units of ethyleneglycol, polyoxyethylene (20) sorbitan monolaurate, polyoxyethylene (20) sorbitan monopalmitate, polyoxyethylene (20) sorbitan monostearate, polyoxyethylene (20) sorbitan monooleate, sorbitan monolaurate, sorbitan monopalmitate, sorbitan monostearate, sorbitan monooleate, ethoxylated hydrogenated ricin oils, sodium laurylsulfate, a diblock copolymer of ethyleneoxyde and propyleneoxyde, Ethylene Oxide-Propylene Oxide Block Copolymers, and tetra-functional block copolymers based on ethylene oxide and propylene oxide, Glyceryl monoesters, Glyceryl caprate, Glyceryl caprylate, Glyceryl cocate, Glyceryl erucate, Glyceryl hydroxysterate, Glyceryl isostearate, Glyceryl lanolate, Glyceryl laurate, Glyceryl linolate, Glyceryl myristate, Glyceryl oleate, Glyceryl PABA, Glyceryl palmitate, Glyceryl ricinoleate, Glyceryl stearate, Glyceryl thiglycolate, Glyceryl dilaurate, Glyceryl dioleate, Glyceryl dimyristate, Glyceryl disterate, Glyceryl sesuioleate, Glyceryl stearate lactate, Polyoxyethylene cetyl/stearyl ether, Polyoxyethylene cholesterol ether, Polyoxyethylene laurate or dilaurate, Polyoxyethylene stearate or distearate, polyoxyethylene fatty ethers, Polyoxyethylene lauryl ether, Polyoxyethylene stearyl ether, polyoxyethylene myristyl ether, a steroid, Cholesterol, Betasitosterol, Bisabolol, fatty acid esters of alcohols, isopropyl myristate, Aliphati-isopropyl n-butyrate, Isopropyl n-hexanoate, Isopropyl n-decanoate, Isoproppyl palmitate, Octyldodecyl myristate, alkoxylated alcohols, alkoxylated acids, alkoxylated amides, alkoxylated sugar derivatives, alkoxylated derivatives of natural oils and waxes, polyoxyethylene polyoxypropylene block copolymers, nonoxynol-14, PEG-8 laurate, PEG-6 Cocoamide, PEG-20 methylglucose sesquistearate, PEG40 lanolin, PEG-40 castor oil, PEG-40 hydrogenated castor oil, polyoxyethylene fatty ethers, glyceryl diesters, polyoxyethylene stearyl ether, polyoxyethylene myristyl ether, and polyoxyethylene lauryl ether, glyceryl dilaurate, glyceryl dimystate, glyceryl distearate, semi-synthetic derivatives thereof, or mixtures thereof.

Nonionic surfactants include, but are not limited to, an ethoxylated surfactant, an alcohol ethoxylated, an alkyl phenol ethoxylated, a fatty acid ethoxylated, a monoalkaolamide ethoxylated, a sorbitan ester ethoxylated, a fatty amino ethoxylated, an ethylene oxide-propylene oxide copolymer, Bis(polyethylene glycol bis[imidazoyl carbonyl]), nonoxynol-9, Bis(polyethylene glycol bis[imidazoyl carbonyl]), Brij® 35, Brij® 56, Brij® 72, Brij® 76, Brij® 92V, Brij® 97, Brij 58P, Cremophor® EL, Decaethylene glycol monododecyl ether, N-Decanoyl-N-methylglucamine, n-Decyl alpha-D-glucopyranoside, Decyl beta-D-maltopyranoside, n-Dodecanoyl-N-methylglucamide, n-Dodecyl alpha-D-maltoside, n-Dodecyl beta-D-maltoside, n-Dodecyl beta-D-maltoside, Heptaethylene glycol monodecyl ether, Heptaethylene glycol monododecyl ether, Heptaethylene glycol monotetradecyl ether, n-Hexadecyl beta-D-maltoside, Hexaethylene glycol monododecyl ether, Hexaethylene glycol monohexadecyl ether, Hexaethylene glycol monooctadecyl ether, Hexaethylene glycol monotetradecyl ether, Igepal CA-630, Igepal CA-630, Methyl-6-O-(N-heptylcarbamoyl)-alpha-D-glucopyranoside, Nonaethylene glycol monododecyl ether, N-N-Nonanoyl-N-methylglucamine, Octaethylene glycol monodecyl ether, Octaethylene glycol monododecyl ether, Octaethylene glycol monohexadecyl ether, Octaethylene glycol monooctadecyl ether, Octaethylene glycol monotetradecyl ether, Octyl-beta-D-glucopyranoside, Pentaethylene glycol monodecyl ether, Pentaethylene glycol monododecyl ether, Pentaethylene glycol monohexadecyl ether, Pentaethylene glycol monohexyl ether, Pentaethylene glycol monooctadecyl ether, Pentaethylene glycol monooctyl ether, Polyethylene glycol diglycidyl ether, Polyethylene glycol ether W-1, Polyoxyethylene 10 tridecyl ether, Polyoxyethylene 100 stearate, Polyoxyethylene 20 isohexadecyl ether, Polyoxyethylene 20 oleyl ether, Polyoxyethylene 40 stearate, Polyoxyethylene 50 stearate, Polyoxyethylene 8 stearate, Polyoxyethylene bis(imidazolyl carbonyl), Polyoxyethylene 25 propylene glycol stearate, Saponin from Quillaja bark, Span® 20, Span® 40, Span® 60, Span® 65, Span® 80, Span® 85, Tergitol, Type 15-S-12, Tergitol, Type 15-S-30, Tergitol, Type 15-S-5, Tergitol, Type 15-S-7, Tergitol, Type 15-S-9, Tergitol, Type NP-10, Tergitol, Type NP-4, Tergitol, Type NP-40, Tergitol, Type NP-7, Tergitol, Type NP-9, Tergitol, Tergitol, Type TMN-10, Tergitol, Type TMN-6, Tetradecyl-beta-D-maltoside, Tetraethylene glycol monodecyl ether, Tetraethylene glycol monododecyl ether, Tetraethylene glycol monotetradecyl ether, Triethylene glycol monodecyl ether, Triethylene glycol monododecyl ether, Triethylene glycol monohexadecyl ether, Triethylene glycol monooctyl ether, Triethylene glycol monotetradecyl ether, Triton CF-21, Triton CF-32, Triton DF-12, Triton DF-16, Triton GR-5M, Triton QS-15, Triton QS-44, Triton X-100, Triton X-102, Triton X-15, Triton X-151, Triton X-200, Triton X-207, Triton® X-114, Triton® X-165, Triton® X-305, Triton® X-405, Triton® X-45, Triton® X-705-70, TWEEN® 20, TWEEN® 21, TWEEN® 40, TWEEN® 60, TWEEN® 61, TWEEN® 65, TWEEN® 80, TWEEN® 81, TWEEN® 85, Tyloxapol, n-Undecyl beta-D-glucopyranoside, semi-synthetic derivatives thereof, or combinations thereof.

In addition, the nonionic surfactant can be a poloxamer. Poloxamers are polymers made of a block of polyoxyethylene, followed by a block of polyoxypropylene, followed by a block of polyoxyethylene. The average number of units of polyoxyethylene and polyoxypropylene varies based on the number associated with the polymer. For example, the smallest polymer, Poloxamer 101, consists of a block with an average of 2 units of polyoxyethylene, a block with an average of 16 units of polyoxypropylene, followed by a block with an average of 2 units of polyoxyethylene. Poloxamers range from colorless liquids and pastes to white solids. In cosmetics and personal care products, Poloxamers are used in the formulation of skin cleansers, bath products, shampoos, hair conditioners, mouthwashes, eye makeup remover and other skin and hair products. Examples of Poloxamers include, but are not limited to, Poloxamer 101, Poloxamer 105, Poloxamer 108, Poloxamer 122, Poloxamer 123, Poloxamer 124, Poloxamer 181, Poloxamer 182, Poloxamer 183, Poloxamer 184, Poloxamer 185, Poloxamer 188, Poloxamer 212, Poloxamer 215, Poloxamer 217, Poloxamer 231, Poloxamer 234, Poloxamer 235, Poloxamer 237, Poloxamer 238, Poloxamer 282, Poloxamer 284, Poloxamer 288, Poloxamer 331, Poloxamer 333, Poloxamer 334, Poloxamer 335, Poloxamer 338, Poloxamer 401, Poloxamer 402, Poloxamer 403, Poloxamer 407, Poloxamer 105 Benzoate, and Poloxamer 182 Dibenzoate.

Suitable anionic surfactants include, but are not limited to, a carboxylate, a sulphate, a sulphonate, a phosphate, chenodeoxycholic acid, chenodeoxycholic acid sodium salt, cholic acid, ox or sheep bile, Dehydrocholic acid, Deoxycholic acid, Deoxycholic acid, Deoxycholic acid methyl ester, Digitonin, Digitoxigenin, N,N-Dimethyldodecylamine N-oxide, Docusate sodium salt, Glycochenodeoxycholic acid sodium salt, Glycocholic acid hydrate, synthetic, Glycocholic acid sodium salt hydrate, synthetic, Glycodeoxycholic acid monohydrate, Glycodeoxycholic acid sodium salt, Glycolithocholic acid 3-sulfate disodium salt, Glycolithocholic acid ethyl ester, N-Lauroylsarcosine sodium salt, N-Lauroylsarcosine solution, N-Lauroylsarcosine solution, Lithium dodecyl sulfate, Lithium dodecyl sulfate, Lithium dodecyl sulfate, Lugol solution, Niaproof 4, Type 4, 1-Octanesulfonic acid sodium salt, Sodium 1-butanesulfonate, Sodium 1-decanesulfonate, Sodium 1-decanesulfonate, Sodium 1-dodecanesulfonate, Sodium 1-heptanesulfonate anhydrous, Sodium 1-heptanesulfonate anhydrous, Sodium 1-nonanesulfonate, Sodium 1-propanesulfonate monohydrate, Sodium 2-bromoethanesulfonate, Sodium cholate hydrate, Sodium choleate, Sodium deoxycholate, Sodium deoxycholate monohydrate, Sodium dodecyl sulfate, Sodium hexanesulfonate anhydrous, Sodium octyl sulfate, Sodium pentanesulfonate anhydrous, Sodium taurocholate, Taurochenodeoxycholic acid sodium salt, Taurodeoxycholic acid sodium salt monohydrate, Taurohyodeoxycholic acid sodium salt hydrate, Taurolithocholic acid 3-sulfate disodium salt, Tauroursodeoxycholic acid sodium salt, Trizma® dodecyl sulfate, TWEEN® 80, Ursodeoxycholic acid, semi-synthetic derivatives thereof, and combinations thereof.

Suitable zwitterionic surfactants include, but are not limited to, an N-alkyl betaine, lauryl amindo propyl dimethyl betaine, an alkyl dimethyl glycinate, an N-alkyl amino propionate, CHAPS, minimum 98% (TLC), CHAPS, minimum 98% (TLC), CHAPS, for electrophoresis, minimum 98% (TLC), CHAPSO, minimum 98%, CHAPSO, CHAPSO, for electrophoresis, 3-(Decyldimethylammonio)propanesulfonate inner salt, 3-Dodecyldimethylammonio)propanesulfonate inner salt, 3-(Dodecyldimethylammonio)propanesulfonate inner salt, 3-(N,N-Dimethylmyristylammonio)propanesulfonate, 3-(N,N-Dimethyloctadecylammonio)propanesulfonate, 3-(N,N-Dimethyloctylammonio)propanesulfonate inner salt, 3-(N,N-Dimethylpalmitylammonio)propanesulfonate, semi-synthetic derivatives thereof, and combinations thereof.

In another embodiment of the invention, the nanoemulsion comprises at least one cationic surfactant and at least one non-cationic surfactant. The non-cationic surfactant is a nonionic surfactant, such as a polysorbate (Tween), such as polysorbate 80 or polysorbate 20. In one embodiment, the non-ionic surfactant is present in a concentration of about 0.05% to about 7.0%, or the non-ionic surfactant is present in a concentration of about 0.3% to about 4%.

### 5. Additional Active Agents

Optionally, a second anti-acne agent is incorporated into the nanoemulsion to achieve better efficacy, tolerability and/or synergistic antimicrobial activity effect in sebum. Preferably, the second anti-acne agent is benzoyl peroxide salicylic acid, or a retinoid. However, any active agent useful in treating acne can be incorporated into the nanoemulsion.

Exemplary topical anti-acne agents include, but are not limited to, benzoyl peroxide, salicylic acid, acitretin, alcloxa, aldioxa, allantoin, dibenzothiophene, etarotent, etretinate, motretinide, nordihydroguaiaretic acid, podofilox, podophyllum resin, resorcinalm resorcinol monoacetate, sumarotene, tetroquinone, adapalene, tretinoin, erythromycin, clindamycin, azelaic acid, hydrocortisone, sodium hyaluronate, sulfur, urea, meclocycline, dapsone, retinoids and retinoid derivatives. Other anti-acne ingredients include Ascorbyl Tetraisopalmitate, Dipotassium Glycyrrhizinate, Ascorbyl Tetraisopalmitate, Niacinamide, alpha bisabolol can also be included in the nanoemulsion of this invention. All of these skin care ingredients have properties that help to reduce and control acne, and acne related problems such as sebum production.

Additional anti-acne agents include acne herbal medicines, such as Tea Tree Oil red clover, lavender, leaves of strawberry, chaste tree berry extract, burdock root, dandelion leaves, milk thistle, papaya enzymes, burdock and dandelion, eucalyptus, thyme, witch hazel, sage oil, camphor, cineole, rosmarinic acid and tannins in the sage oil.

### 6. Additional Ingredients

Additional compounds suitable for use in the nanoemulsions of the invention include but are not limited to one or more solvents, such as an organic phosphate-based solvent, bulking agents, coloring agents, pharmaceutically acceptable excipients, a preservative, pH adjuster, buffer, chelating agent, etc. The additional compounds can be admixed into a previously emulsified nanoemulsion, or the additional compounds can be added to the original mixture to be emulsified. In certain of these embodiments, one or more additional compounds are admixed into an existing nanoemulsion composition immediately prior to its use.

Suitable preservatives in the nanoemulsions of the invention include, but are not limited to, cetylpyridinium chloride, benzalkonium chloride, benzyl alcohol, chlorhexidine, imidazolidinyl urea, phenol, potassium sorbate, benzoic acid, bronopol, chlorocresol, paraben esters, phenoxyethanol, sorbic acid, alpha-tocophernol, ascorbic acid, ascorbyl palmitate, butylated hydroxyanisole, butylated hydroxytoluene, sodium ascorbate, sodium metabisulphite, citric acid, edetic acid, semi-synthetic derivatives thereof, and combinations thereof.

The nanoemulsion may further comprise at least one pH adjuster. Suitable pH adjusters in the nanoemulsion of the invention include, but are not limited to, diethyanolamine, lactic acid, monoethanolamine, triethylanolamine, sodium hydroxide, sodium phosphate, semi-synthetic derivatives thereof, and combinations thereof.

In addition, the nanoemulsion can comprise a chelating agent. In one embodiment of the invention, the chelating agent is present in an amount of about 0.0005% to about 1.0%. Examples of chelating agents include, but are not limited to, ethylenediamine, ethylenediaminetetraacetic acid (EDTA), and dimercaprol, and a preferred chelating agent is ethylenediaminetetraacetic acid.

The nanoemulsion can comprise a buffering agent, such as a pharmaceutically acceptable buffering agent. Examples of buffering agents are disclosed in U.S. Patent Publication No. 2010/0226983

The nanoemulsion can comprise one or more emulsifying agents to aid in the formation of emulsions. Emulsifying agents include compounds that aggregate at the oil/water interface to form a kind of continuous membrane that prevents direct contact between two adjacent droplets. Certain embodiments of the present invention feature nanoemulsions that may readily be diluted with water to a desired concentration without impairing their anti-fungal or antiyeast properties.

### D. Pharmaceutical Compositions

The nanoemulsions of the invention may be formulated into pharmaceutical compositions that comprise the nanoemulsion in a therapeutically effective amount and suitable, pharmaceutically-acceptable excipients for topical administration to a human subject in need thereof. Such excipients are well known in the art.

By the phrase “therapeutically effective amount” it is meant any amount of the nanoemulsion that is effective in preventing and/or treating acne. One possible way to treat acne is by killing or inhibiting the growth of P. acnes, causing P. acnes to lose pathogenicity, or any combination thereof.

Topical administration includes administration to the skin, including surface of the hair follicle and pilosebaceous unit.

Pharmaceutically acceptable dosage forms for topical administration include, but are not limited to, ointments, creams, liquids, emulsions, lotions, gels, bioadhesive gels, aerosols, pastes, foams, sunscreens, or in the form of an article or carrier, such as a bandage, insert, syringe-like applicator, pessary, powder, talc or other solid, cleanser (leave on and wash off product), and agents that favor penetration within the pilosebaceous gland.

The pharmaceutical compositions may be formulated for immediate release, sustained release, controlled release, delayed release, or any combinations thereof, into the epidermis or dermis, with no systemic absorption. In some embodiments, the formulations may comprise a penetration-enhancing agent for enhancing penetration of the nanoemulsion through the stratum corneum and into the epidermis or dermis. Suitable penetration-enhancing agents include, but are not limited to, alcohols such as ethanol, triglycerides and aloe compositions. The amount of the penetration-enhancing agent may comprise from about 0.5% to about 40% by weight of the formulation.

In some embodiments, the formulation for delivery via a “patch” comprising a therapeutically effective amount of the nanoemulsion is envisioned. As used herein a “patch” comprises at least a topical formulation and a covering layer, such that the patch can be placed over the area to be treated. Preferably, the patch is designed to maximize delivery through the stratum corneum and into the epidermis or dermis, while minimizing absorption into the circulatory system, and little to no skin irritation, reducing lag time, promoting uniform absorption, and reducing mechanical rub-off and dehydration.

Adhesives for use with the drug-in-adhesive type patches are well known in the art. Suitable adhesive include, but are not limited to, polyisobutylenes, silicones, and acrylics. These adhesives can function under a wide range of conditions, such as, high and low humidity, bathing, sweating etc. Preferably the adhesive is a composition based on natural or synthetic rubber; a polyacrylate such as, polybutylacrylate, polymethylacrylate, poly-2-ethylhexyl acrylate; polyvinylacetate; polydimethylsiloxane; or and hydrogels (e.g., high molecular weight polyvinylpyrrolidone and oligomeric polyethylene oxide). The most preferred adhesive is a pressure sensitive acrylic adhesive, for example Durotak® adhesives (e.g., Durotak® 2052, National Starch and Chemicals). The adhesive may contain a thickener, such as a silica thickener (e.g., Aerosil, Degussa, Ridgefield Park, N.J.) or a crosslinker such as aluminumacetylacetonate.

Suitable release liners include but are not limited to occlusive, opaque, or clear polyester films with a thin coating of pressure sensitive release liner (e.g., silicone-fluorsilicone, and perfluorcarbon based polymers.

Backing films may be occlusive or permeable and are derived from synthetic polymers like polyolefin oils polyester, polyethylene, polyvinylidine chloride, and polyurethane or from natural materials like cotton, wool, etc. Occlusive backing films, such as synthetic polyesters, result in hydration of the outer layers of the stratum corneum while non-occlusive backings allow the area to breath (i.e., promote water vapor transmission from the skin surface). More preferably the backing film is an occlusive polyolefin foil (Alevo, Dreieich, Germany). The polyolefin foil is preferably about 0.6 to about 1 mm thick.

The shape of the patch can be flat or three-dimensional, round, oval, square, and have concave or convex outer shapes, or the patch or bandage can also be segmented by the user into corresponding shapes with or without additional auxiliary means.

The pharmaceutical compositions for topical administration may be applied in a single administration or in multiple administrations. The pharmaceutical compositions are topically applied for at least once a week, at least twice a week, at least once a day, at least twice a day, multiple times daily, multiple times weekly, biweekly, at least once a month, or any combination thereof. The pharmaceutical compositions are topically applied for a period of time of about one month, about two months, about three months, about four months, about five months, about six months, about seven months, about eight months, about nine months, about ten months, about eleven months, about one year, about 1.5 years, about 2 years, about 2.5 years, about 3 years, about 3.5 years, about 4 years, about 4.5 years, and about 5 years. Between applications, the application area may be washed to remove any residual nanoemulsion.

Preferably, the pharmaceutical compositions are applied to the skin area in an amount of from about 0.001 mL/cm² to about 5.0 mL/cm². An exemplary application amount and area is about 0.2 mL/cm², although any amount from 0.001 mL/cm² up to about 5.0 mL/cm² can be applied. Following topical administration, the nanoemulsion may be occluded or semi-occluded. Occlusion or semi-occlusion may be performed by overlaying a bandage, polyoleofin film, impermeable barrier, or semi-impermeable barrier to the topical preparation. Preferably, after application, the treated area is covered with a dressing.

### E. Methods of Manufacture

The nanoemulsions of the invention can be formed using classic emulsion forming techniques. See e.g., U.S. 2004/0043041. See also the method of manufacturing nanoemulsions described in U.S. Pat. Nos. 6,559,189, 6,506,803, 6,635,676, 6,015,832, and U.S. Patent Publication Nos. 20040043041, 20050208083, 20060251684, and 20070036831, and WO 05/030172, all of which are specifically incorporated by reference. In an exemplary method, the oil is mixed with the aqueous phase under relatively high shear forces (e.g., using high hydraulic and mechanical forces) to obtain a nanoemulsion comprising oil droplets having an average diameter of less than about 1000 nm. Some embodiments of the invention employ a nanoemulsion having an oil phase comprising an alcohol such as ethanol. The oil and aqueous phases can be blended using any apparatus capable of producing shear forces sufficient to form an emulsion, such as French Presses or high shear mixers (e.g., FDA approved high shear mixers are available, for example, from Admix, Inc., Manchester, N.H.). Methods of producing such emulsions are described in U.S. Pat. Nos. 5,103,497 and 4,895,452, herein incorporated by reference in their entireties.

In an exemplary embodiment, the nanoemulsions used in the methods of the invention comprise droplets of an oily discontinuous phase dispersed in an aqueous continuous phase, such as water. The nanoemulsions of the invention are stable, and do not decompose even after long storage periods. Certain nanoemulsions of the invention are non-toxic and safe when swallowed, inhaled, or contacted to the skin of a subject.

The compositions of the invention can be produced in large quantities and are stable for many months at a broad range of temperatures. The nanoemulsion can have textures/consistencies ranging from that of a semi-solid cream to that of a thin lotion and can be applied topically by hand and sprayed onto a surface. As stated above, at least a portion of the emulsion may be in the form of lipid structures including, but not limited to, unilamellar, multilamellar, and paucliamellar lipid vesicles, micelles, and lamellar phases.

The nanoemulsion of the invention can be provided in many different types of containers and delivery systems. For example, in some embodiments of the invention, the nanoemulsions are provided in a cream or other solid or semi-solid form. The nanoemulsions of the invention may be incorporated into hydrogel formulations.

The nanoemulsions can be delivered (e.g., to a subject or customers) in any suitable container. Suitable containers can be used that provide one or more single use or multi-use dosages of the nanoemulsion for the desired application. In some embodiments of the invention, the nanoemulsions are provided in a suspension or liquid form. Such nanoemulsions can be delivered in any suitable container including spray bottles (e.g., pressurized spray bottles).

### F. EXAMPLES

The invention is further described by reference to the following examples, which are provided for illustration only. The invention is not limited to the examples, but rather includes all variations that are evident from the teachings provided herein. All publicly available documents referenced herein, including but not limited to U.S. patents and published patent applications, are specifically incorporated by reference.

### Example 1

Nanoemulsions are produced by mixing a water-immiscible oil phase into an aqueous phase with a proprietary manufacturing method. The two phases (aqueous phase and oil phase) are combined together and processed to yield an emulsion. The emulsion is further processed to achieve the desired particle size. For a gel formulation, a thickening agent, such as Klucel can be added to the nanoemulsion. For example, Klucel is dissolved in water or any aqueous solvent and added to the nanoemulsion to achieve the desired concentration.

Since nanoemulsions are not a single small molecule, their relative activity can be expressed in terms of the concentration of cationic surfactant present, e.g., CPC or benzalkonium chloride.

NB-003 is an exemplary nanoemulsion comprising, in an aqueous medium, different amounts of the quaternary ammonium compound CPC, soybean oil, Tween 20®, ethanol, ethylenediaminetetraacetic acid (EDTA) and water. The ingredients of the nanoemulsions used in the examples below are shown in Table 1. The percentages are wt/wt, unless otherwise noted.

TABLE 1
Compositions of the Nanoemulsions used in the examples below.

| | | | | CPC | | |
|---|---|---|---|---|---|---|
| Formulation | Soybean oil % | Tween 20 % | Ethanol % | % (w/v) | EDTA % | Water % |
| 0.01% NB-003 | 0.6279 | 0.0592 | 0.0679 | 0.0107 | 0.00074 | 99.24 |
| 0.1% NB-003 | 6.279 | 0.592 | 0.679 | 0.107 | 0.0074 | 92.34 |
| 0.25% NB-003 | 15.697 | 1.48 | 1.698 | 0.267 | 0.0185 | 80.84 |
| 0.3% NB-003 | 18.837 | 1.776 | 2.037 | 0.320 | 0.022 | 77.01 |
| 0.5% NB-003 | 31.394 | 2.96 | 3.395 | 0.534 | 0.037 | 61.68 |
| 0.8% NB-003 | 50.240 | 4.736 | 5.384 | 0.854 | 0.059 | 38.73 |

### Example 2

The purpose of this example was to evaluate the bactericidal effect on pig skin inoculated with P. acnes following application of four different nanoemulsions varying in their concentration of CPC.

Following inoculation of pig skin with P. acnes, the pig skin was treated with four different nanoemulsion formulations, which differed in their concentration of CPC: (1) 0.01% NB-003; (2) 0.1% NB-003; (3) 0.25% NB-003; and (4) 0.5% NB-003. After 1 hour of treatment, the log reduction of P. acnes bacterial colonies was measured. As shown in FIG. 2, all concentrations of NB-oo3 tested were found to have high bactericidal activity in the preclinical skin model. No substantial difference was seen in the bactericidal results of the four different nanoemulsion concentrations tested.

### Example 3

The purpose of this example was to evaluate in vivo the efficacy of a nanoemulsion comprising 0.5% CPC in treating and/or preventing acne in human subjects. Effectiveness was evaluated by quantitative microbiologic determinations of P. acnes levels before, during and after treatment in healthy human volunteers. In a smaller group, the effects of vehicle were measured for comparative efficacy.

P. acnes are the most common gram-positive microaerophilic organisms found on normal skin. Although it has no intrinsic pathogenicity, P. acnes is believed to play a major role in the pathogenesis of acne. Most presently available topical anti-acne preparations such as benzoyl peroxides and topical antimicrobials exert their therapeutic effect through inhibition of P. acnes in vivo as demonstrated by a 1.0 to 2.0 logarithmic colony reduction.

Study subjects comprised healthy adult males and females 18 years of age and older with high levels of P. acnes colonization (10,000 colonies per cm²) on the forehead. Twenty-five subjects received NB-003 0.5% (5.0 mg/mL cetylpyridinium chloride, CPC) and ten subjects received vehicle. Of the 35 subjects enrolled, 30 subjects completed the trial.

Treatment was for 4 weeks. For the NB-003 treatments, 2 mL of product was applied to a cosmetic cotton pad, followed by repeated swiping across the forehead. Approximately 0.3 to 0.5 mL volume of nanoemulsion or vehicle was applied to the entire forehead area twice daily.

Quantitative Bacteriology: Quantitative bacteriologic cultures were obtained at Screening, Baseline, Weeks 1, 2 and 4. Samples were obtained by cleansing the forehead of surface bacteria by thoroughly wiping the area for 30 seconds with sterile gauze soaked with 0.1% Triton-X-100 to remove surface debris and bacteria. The surface areas cultured (3.8 cm²) were then delineated by a sterile plastic template held firmly to the skin. A sterile cotton-tipped swab was dipped into 2 ml of wash solution (Bacto Letheen Broth, Difco, Sparks, Md., USA). The area was then scrubbed with the cotton-tipped swab for 30 seconds. The swab was then placed back into the 2 ml wash solution and wrung on the side of the tube. The same skin areas were then scrubbed again for another 30 seconds after which the cotton-tipped swab is again placed back into the 2 ml of wash solution and wrung on the side of the tube. The swab was then broken off into the 2 ml of wash solution. The sample was subsequently processed as described in the Williamson-Kligman method. The wash sample was serially diluted using 0.05% Tween-80 (buffered with 0.075M phosphate buffer, pH 7.9) in 4 ten-fold dilutions.

Using a micropipettor, 0.05 mL of each dilution was placed on a designated section of an agar plate containing Brucella agar supplemented with yeast extract, dextrose, and cysteine, five-drop dilutions per plate. Plates were allowed to dry, placed in an anaerobic jar with BBL Gas Pak Plus anaerobic system envelope and incubated anaerobically at 36.8-37.2° C. for 7 days. Colony forming units (cfu) of P. acnes were counted at the dilution that contains between 10 and 100 cfu. Total densities of P. acnes are calculated and reported as log 10 cfu per cm².

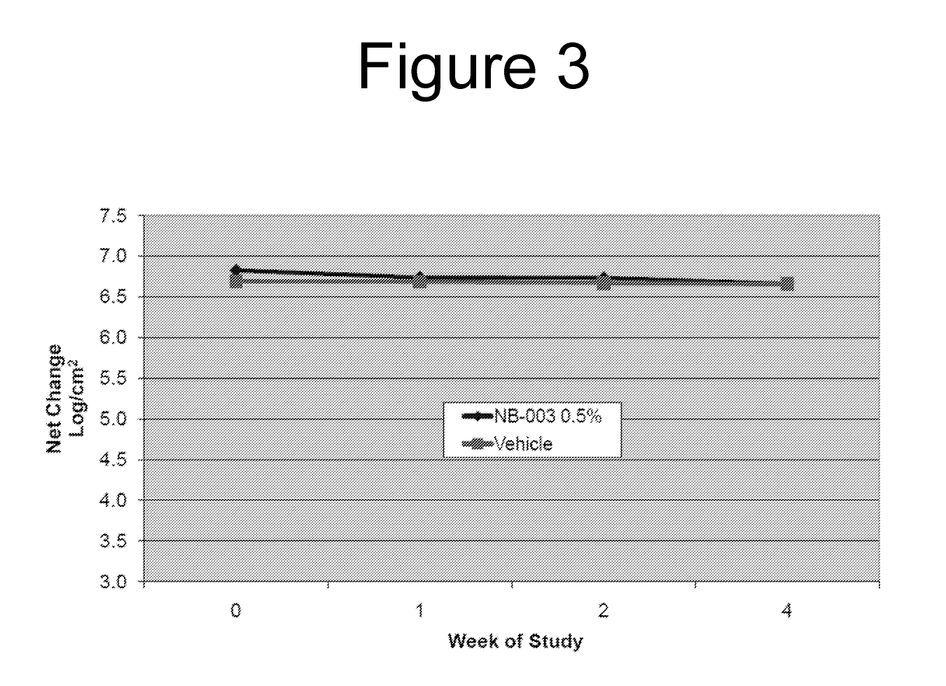

Results: No adverse events relating to the study medication were reported. The tolerance and in-vivo antimicrobial effects of NB-003 0.5% and vehicle on P. acnes colonization of sebaceous follicles were measured. Both formulations were well tolerated. NB-003 0.5% produced a 0.2 log reduction in P. acnes after 4 weeks of treatment. There were a handful of subjects who showed a 0.3 to 1.0 logarithm reduction, but overall there was no increase in activity from that seen with vehicle. See FIG. 3.

### Example 4

The purpose of this investigation was to evaluate the safety and efficacy of NB-003 (0.1% and 0.3%) by quantitative microbiologic determinations of P. acnes levels before, during and after treatment in healthy human subjects.

Study subjects comprised healthy adult males and females 18 years of age and older with high levels of P. acnes colonization (10,000 colonies per cm²) on the forehead. Each subject received one of the following treatments: 0.1% NB-003 (liquid) and 0.3% NB-003 (liquid).

All test articles were applied twice daily to the forehead for a total of 4 weeks. Each subject had one of the two applied to the forehead in accordance with a computer-generated randomization scheme. The design of the randomization allowed for twenty subjects to receive 0.3% NB-003 (3.0 mg/mL cetylpyridinium chloride, CPC) and ten subjects to receive 0.1% NB-003 (1.0 mg/mL CPC).

Treatment was for 4 weeks. Each volunteer was treated twice daily. For the NB-003 treatments, 2 mL of product was applied to a cosmetic cotton pad, and the [symbol] . The saturated pad was repeatedly swiped across the forehead ensuring that the product thoroughly covered the entire forehead. resulting in an application of approximately 0.3 to 0.5 mL volume to the entire forehead area

Quantitative bacteriologic cultures were obtained as described in Example 3.

Results: There were no reports of severe symptoms related to the treatment medication and no subjects withdrew from the study due to dermal irritation.

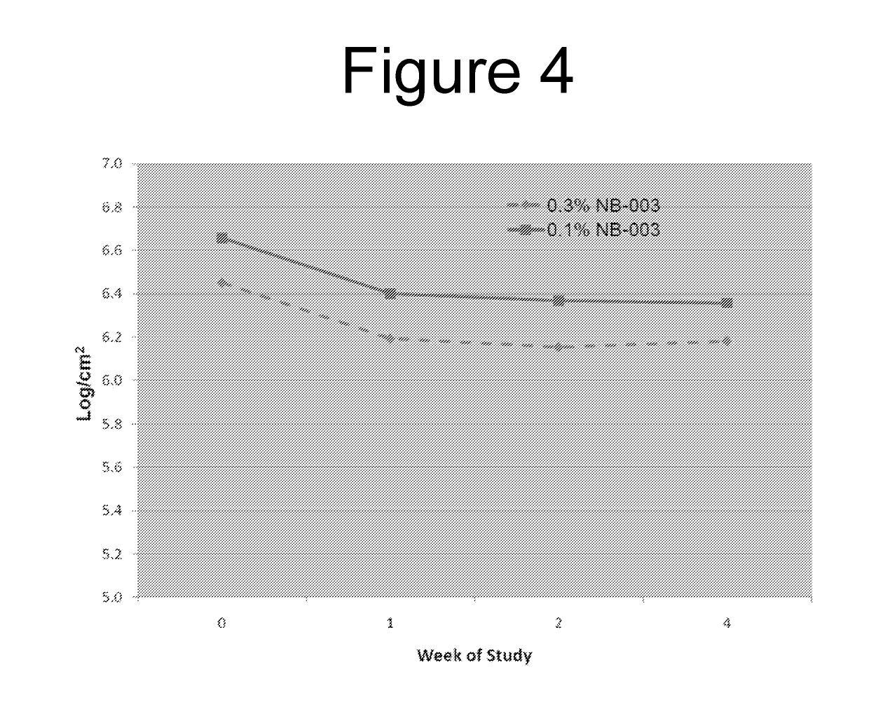

In this study, the effects of 2 concentrations of NB-003 (0.1% and 0.3%) on in-vivo P. acnes levels were measured. Quantitative cultures were obtained at Baseline and after 1, 2 and 4 weeks in 20 individuals treated with 0.3% and 9 treated with 0.1%. Both treatments produced approximately a 0.3 log reduction after 1 week with no further mean reduction over the ensuing weeks of treatment. In the group treated with 0.3% NB-003, approximately 25% showed a greater reduction of a 0.5 or greater log reduction. See FIG. 4. This kind of variability is similar to that seen with topical erythromycin formulations and is suggestive of varying degrees of penetration into sebaceous follicles.

### Example 5

The purpose of this example was to compare the safety and efficacy of four different nanoemulsion formulations differing in their concentration of CPC as compared to vehicle, by quantitative microbiologic determinations of P. acnes levels before and after treatment in healthy human subjects.

In randomized, active-controlled, open label, single-site studies in normal volunteers with P. acnes colonization, subjects were treated twice a day for 4 weeks with one of the following five compositions: (1) vehicle; (2) 0.1% NB-003, (3) 0.25% NB-003, (4) 0.3% NB-003, and (5) 0.5% NB-003. Samples for P. acnes counts were taken at baseline and each week.

Quantitative bacteriologic cultures were obtained as described in Example 3. The results, shown in FIG. 5, are summarized in the table below.

TABLE 2

| | | 0.10% | 0.25% | 0.30% | 0.50% |
|---|---|---|---|---|---|
| Composition | Vechicle | NB-003 | NB-003 | NB-003 | NB-003 |
| Log Reduction | 0.09 | 0.27 | 0.66 | 0.3 | 0.09 |
| P. acnes | | | | | |

Agents with a 1 logarithm base 10 or greater reduction have uniformly been shown to be effective in acne treatment. Thus, based on these results, human bactericidal activity unexpectedly drops off with use of a nanoemulsion having a CPC concentration of 0.5%. The optimal CPC concentration for a nanoemulsion used in treating or preventing acne is therefore about 0.1% CPC up to about 0.4% CPC.

These results are surprising as in preclinical skin models using non-human skin, e.g., pig skin inoculated with P. acnes, all concentrations of nanoemulsions tested—0.01% CPC, 0.1% CPC, 0.25% CPC, and 0.5% CPC—were found to have high bactericidal activity. See FIG. 2 and Example 2 above. In stark contrast, however, these preclinical results were not predictive of the nanoemulsion formulations found to be effective in vivo in treating and/or preventing human acne. Specifically, FIG. 5 and Example 5 show that in in vivo human use, bactericidal activity against P. acnes drops off dramatically at 0.5%, with an optimal CPC concentration between about 0.1% and about 0.4%.

It will be apparent to those skilled in the art that various modifications and variations can be made in the methods and compositions of the present invention without departing from the spirit or scope of the invention. Thus, it is intended that the present invention cover the modifications and variations of this invention provided they come within the scope of the appended claims and their equivalents.

1. A method of treating and/or preventing acne by reducing the amount of P. acnes present in a human subject in need thereof, wherein the method comprises topically administering to the subject a nanoemulsion, wherein:
(a) the nanoemulsion comprises droplets having an average diameter of less than about 3 microns;
(b) the nanoemulsion droplets comprise an oil phase with at least one oil, and an aqueous phase comprising at least one cationic surfactant, at least one organic solvent, and water, wherein the cationic surfactant is:
(i) cetylpyridinium chloride (CPC) is present at a concentration of about 0.1% up to about 0.4%; or
(ii) benzalkonium chloride (BKC) is present at a concentration of about 0.05% up to about 1.6%.

2. The method of claim 1, wherein:
(a) the nanoemulsion droplets target the pilosebaceous gland; and/or
(b) the nanoemulsion droplets enter the pilosebaeous gland (unit), hair follicle, epidermis, dermis, or a combination thereof.

3. The method of claim 1, wherein the nanoemulsion is at room temperature at the time of administration.

4. The method of claim 1, wherein prior to application the nanoemulsion is warmed to a temperature selected from the group consisting of about 30° C. or warmer, about 31° C. or warmer, about 32° C. or warmer, about 33° C. or warmer, about 34° C. or warmer, about 35° C. or warmer, about 36° C. or warmer, and about 37° C.

5. The method of claim 1, wherein CPC is present at a concentration selected from the group consisting of about 0.10%, about 0.11%, about 0.12%, about 0.13%, about 0.14%, about 0.15%, about 0.16%, about 0.17%, about 0.18%, about 0.19%, about 0.20%, about 0.21%, about 0.22%, about 0.23%, about 0.24%, about 0.25%, about 0.26%, about 0.27%, about 0.28%, about 0.29%, about 0.30%, about 0.31%, about 0.32%, about 0.33%, about 0.34%, about 0.35%, about 0.36%, about 0.37%, about 0.38%, about 0.39%, and about 0.4%.

6. The method of claim 1, wherein BKC is present at a concentration selected from the group consisting of about 0.05%, about 0.06%, about 0.07%, about 0.08%, about 0.09%, about 0.10%, about 0.11%, about 0.12%, about 0.13%, about 0.14%, about 0.15%, about 0.16%, about 0.17%, about 0.18%, about 0.19%, about 0.20%, about 0.25%, about 0.3%, about 0.35%, about 0.4%, about 0.45%, about 0.5%, about 0.55%, about 0.6%, about 0.65%, about 0.7%, about 0.75%, about 0.8%, about 0.85%, about 0.9%, about 0.95%, about 1.0%, about 1.05%, about 1.1%, about 1.15%, about 1.2%, about 1.25%, about 1.3%, about 1.35%, about 1.4%, about 1.45%, about 1.5%, about 1.55%, and about 1.6%.

7. The method of claim 1, wherein the nanoemulsion further comprises a chelating agent.

8. The method of claim 7, wherein the chelating agent:
(a) is present in an amount of about 0.0005% to about 1.0%;
(b) is selected from the group consisting of ethylenediamine, ethylenediaminetetraacetic acid, and dimercaprol; or
(c) any combination thereof.

9. The method of claim 1, wherein the nanoemulsion further comprises at least one non-cationic surfactant.

10. The method of claim 9, wherein:
(a) the non-cationic surfactant is a nonionic surfactant;
(b) the non-cationic surfactant is a nonionic surfactant which is is a polysorbate;
(c) the non-cationic surfactant is a nonionic surfactant which is polysorbate 20 or polysorbate 80 or polysorbate 60; and/or
(d) the non-cationic surfactant is a nonionic surfactant and the non-ionic surfactant is present in a concentration of about 0.001% to about 10%.

11. The method of claim 1, wherein the nanoemulsion comprises:
(a) an aqueous phase;
(b) about 1% oil to about 80% oil;
(c) about 0.1% organic solvent to about 50% organic solvent;
(d) about 0.1% to about 0.4% CPC, or about 0.05% to about 1.6% BKC.

12. The method of claim 1, wherein:
(a) the nanoemulsion droplets have an average diameter selected from the group consisting of less than about 950 nm, less than about 900 nm, less than about 850 nm, less than about 800 nm, less than about 750 nm, less than about 700 nm, less than about 650 nm, less than about 600 nm, less than about 550 nm, less than about 500 nm, less than about 450 nm, less than about 400 nm, less than about 350 nm, less than about 300 nm, less than about 250 nm, less than about 200 nm, less than about 150 nm, less than about 100 nm, less than about 75 nm, greater than about 50 nm greater than about 70 nm, greater than about 125 nm, and any combination thereof;
(b) the nanoemulsion droplets have an average diameter greater than about 125 nm and less than about 450 nm; or
(c) any combination thereof.

13. The method of claim 1, wherein the topical application is to any superficial skin structure.

14. The method of claim 1, wherein the organic solvent:
(a) is selected from the group consisting of C₁-C₁₂ alcohol, diol, triol, dialkyl phosphate, tri-alkyl phosphate, semi-synthetic derivatives thereof, and combinations thereof;
(b) is an alcohol which is selected from the group consisting of a nonpolar solvent, a polar solvent, a protic solvent, and an aprotic solvent;
(c) is selected from the group consisting of ethanol, methanol, isopropyl alcohol, glycerol, medium chain triglycerides, diethyl ether, ethyl acetate, acetone, dimethyl sulfoxide (DMSO), acetic acid, n-butanol, butylene glycol, perfumers alcohols, isopropanol, n-propanol, formic acid, propylene glycols, glycerol, sorbitol, industrial methylated spirit, triacetin, hexane, benzene, toluene, diethyl ether, chloroform, 1,4-dixoane, tetrahydrofuran, dichloromethane, acetone, acetonitrile, dimethylformamide, dimethyl sulfoxide, formic acid, semi-synthetic derivatives thereof, and any combination thereof; or
(d) any combination thereof.

15. The method of claim 1, wherein the oil:
(a) is any cosmetically or pharmaceutically acceptable oil:
(b) is non-volatile;
(c) is selected from the group consisting of animal oil, vegetable oil, natural oil, synthetic oil, hydrocarbon oils, silicone oils, and semi-synthetic derivatives thereof;
(d) is selected from the group consisting of mineral oil, squalene oil, flavor oils, silicon oil, essential oils, water insoluble vitamins, Isopropyl stearate, Butyl stearate, Octyl palmitate, Cetyl palmitate, Tridecyl behenate, Diisopropyl adipate, Dioctyl sebacate, Menthyl anthranhilate, Cetyl octanoate, Octyl salicylate, Isopropyl myristate, neopentyl glycol dicarpate cetols, Ceraphyls®, Decyl oleate, diisopropyl adipate, C₁₂₋₁₅ alkyl lactates, Cetyl lactate, Lauryl lactate, Isostearyl neopentanoate, Myristyl lactate, Isocetyl stearoyl stearate, Octyldodecyl stearoyl stearate, Hydrocarbon oils, Isoparaffin, Fluid paraffins, Isododecane, Petrolatum, Argan oil, Canola oil, Chile oil, Coconut oil, corn oil, Cottonseed oil, flaxseed oil, Grape seed oil, Mustard oil, Olive oil, Palm oil, Palm kernel oil, Peanut oil, Pine seed oil, Poppy seed oil, Pumpkin seed oil, Rice bran oil, Safflower oil, Tea oil, Truffle oil, Vegetable oil, Apricot (kernel) oil, Jojoba oil (simmondsia chinensis seed oil), Grapeseed oil, Macadamia oil, Wheat germ oil, Almond oil, Rapeseed oil, Gourd oil, Soybean oil, Sesame oil, Hazelnut oil, Maize oil, Sunflower oil, Hemp oil, Bois oil, Kuki nut oil, Avocado oil, Walnut oil, Fish oil, berry oil, allspice oil, juniper oil, seed oil, almond seed oil, anise seed oil, celery seed oil, cumin seed oil, nutmeg seed oil, leaf oil, basil leaf oil, bay leaf oil, cinnamon leaf oil, common sage leaf oil, eucalyptus leaf oil, lemon grass leaf oil, melaleuca leaf oil, oregano leaf oil, patchouli leaf oil, peppermint leaf oil, pine needle oil, rosemary leaf oil, spearmint leaf oil, tea tree leaf oil, thyme leaf oil, wintergreen leaf oil, flower oil, chamomile oil, clary sage oil, clove oil, geranium flower oil, hyssop flower oil, jasmine flower oil, lavender flower oil, manuka flower oil, Marhoram flower oil, orange flower oil, rose flower oil, ylang-ylang flower oil, Bark oil, cassia Bark oil, cinnamon bark oil, sassafras Bark oil, Wood oil, camphor wood oil, cedar wood oil, rosewood oil, sandalwood oil), rhizome (ginger) wood oil, resin oil, frankincense oil, myrrh oil, peel oil, bergamot peel oil, grapefruit peel oil, lemon peel oil, lime peel oil, orange peel oil, tangerine peel oil, root oil, valerian oil, Oleic acid, Linoleic acid, Oleyl alcohol, Isostearyl alcohol, semi-synthetic derivatives thereof, and combinations thereof; or
(e) any combination thereof.

16. The method of claim 1, wherein the nanoemulsion further comprises:
(a) at least one preservative;
(b) at least one a pH adjuster;
(c) at least pharmaceutically acceptable buffer; or
(d) any combination thereof.

17. The method of claim 1, wherein the water is present in Phosphate Buffered Saline (PBS).

18. The method of claim 1, wherein the nanoemulsion is topically applied:
(a) in a single administration;
(b) for at least once a week, at least twice a week, at least once a day, at least twice a day, multiple times daily, multiple times weekly, biweekly, at least once a month, or any combination thereof;
(c) for a period of time selected from the group consisting of about one week, about two weeks, about three weeks, about one month, about two months, about three months, about four months, about five months, about six months, about seven mouths, about eight months, about nine months, about ten months, about eleven months, about one year, about 1.5 years, about 2 years, about 2.5 years, about 3 years, about 3.5 years, about 4 years, about 4.5 years, and about 5 years;
(d) followed by washing the application area to remove any residual nanoemulsion; or
(e) any combination thereof.

19. The method of claim 1, wherein the nanoemulsion further comprises at least one anti-acne agent.

20. The method of claim 19, wherein the anti-acne agent is selected from the group consisting of benzoyl peroxide, salicylic acid and retinoid.
